(19) United States
(12) Patent Application Publication
(10) Pub. No.: US 2005/0215974 A1
(43) Pub. Date: Sep. 29, 2005

(54) Absorbent garment with integral elastomeric side panels

(76) Inventor: Susan O'Connell, Coronado, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/212,008

(22) Filed: Aug. 2, 2002

Related U.S. Application Data
(60) Provisional application No. 60/311,367, filed on Aug. 13, 2001.

(51) Int. Cl. A61F 13/15

(52) U.S. Cl. 604/392

(57) ABSTRACT

The invention generally relates to an integral elastomeric side panel that is suitable for use in or on a disposable absorbent garment. The integral elastomeric side panel is formed from a material that has unidirectional or multidirectional stretch features enabling it to stretch in the lateral, or longitudinal and lateral directions. A fastening device is associated with the integral elastomeric side panel. A disposable absorbent garment including the integral elastomeric side panel also is disclosed. The integral elastomeric side panel is formed from the unitary composite sheet that is prepared during assembly of the absorbent garment in a manner such that it does not require separate attachment to the garment. A rear waist edge of the absorbent garment, while in a relaxed state, can extend a length that is greater than the lateral dimension of the unitary composite sheet from which the garment is formed.

### CROSS REFERENCE TO RELATED APPLICATIONS

This is a continuation of application Ser. No. 10/212,008 filed Aug. 2, 2002.

### FIELD OF THE INVENTION

The invention generally relates to an integral elastomeric side panel that is suitable for use in or on a disposable absorbent garment. The integral elastomeric side panel is formed from an elastomeric material that has unidirectional or multidirectional stretch features enabling it to stretch in the lateral or lateral and longitudinal direction of the absorbent garment. A fastening device is associated with the integral elastomeric side panel. The integral elastomeric side panels are formed from a unitary composite sheet that is also used to form the absorbent garment, and the length of the rear waist edge of the absorbent garment may be greater than the lateral width of the unitary composite sheet. A disposable absorbent garment including the integral elastomeric side panels, and a method for producing the disposable absorbent garment, is also disclosed.

### BACKGROUND OF THE INVENTION

Absorbent garments, such as diapers and adult incontinence products, often are constructed to have an hourglass or rectangular shape that is fitted between a wearer's legs, wrapped around the wearer's waist, and then sealed on either side to form a pant-like structure on the wearer. Many of these garments have an elasticized waist that the caregiver stretches out while placing the garment on the wearer. The waist portion that typically is stretched is that portion normally associated with the sides of a user. Thus, when the caregiver is placing the garment on the user, the side portions of the waist can be stretched around to the front waist portion and attached thereto to complete the waist of the garment.

Often, the narrower portion of the known absorbent garments is formed by trimming a portion of the unitary composite sheet or layers that are assembled into the unitary composite sheet, such as the top sheet or back sheet, to provide a contour for leg openings in the finished garment. These cutouts are adapted to be placed between the legs of the wearer with the wider portions of the garment being adapted to encircle the waist of a wearer so that the front and rear portions overlap and can be easily attached to one another. Recently, it has become desirable to produce absorbent garments, such as infant diapers, that fit more closely to the body of a wearer. Accordingly, it has become desirable to make such garments less bulky in use while still maintaining a high level of absorbent protection.

Leg openings are defined, in part, on traditional infant diapers by the overlapped front and rear portions of the waist area of the diaper. Garments with such leg openings are generally perceived as providing good absorbent protection. In the past, known fastening devices for absorbent garments have consisted of substantially inelastic devices to hold the garment in place around the waist of a wearer which can lead to undesirable red marking of the skin of a wearer if stretched too tightly. More recent advancements include incorporating elastic portions in the waist area to maintain fit and comfort, and reduce red markings.

Introducing a degree of stretchability into personal care products such as diapers, sanitary napkins, adult incontinent products and the like is believed to provide these products with certain benefits. For example, U.S. Pat. No. 2,866,459 describes a stretchable, reusable cotton diaper. Stretchability is imparted to the cotton fabric by the presence of longitudinal and transverse elastic stitching. U.S. Pat. No. 3,371,668 discloses a sanitary napkin employing a nonwoven fabric with machine direction elasticity as a cover. The described fabric comprises a nonwoven web of fibers having a plurality of elastic means, in extended condition, secured to the web.

U.S. Pat. No. 4,606,964 describes a bulked web composite and method of making the same, where the composite is formed by adhering a reticulated web of elastic material to at least one gatherable web. Upon release of the tensioning forces, the gatherable web is gathered by the reticulated web to form a bulked web composite.

U.S. Pat. No. 4,847,134 discloses a stretchable absorbent undergarment, including an inner layer or top sheet, a liquid impervious outer layer or back sheet, an absorbent layer and a stretchable layer. The stretchable layer comprises a continuous layer of stretchable material that may be perforated. These perforations are said to render the elastomeric layer permeable.

U.S. Pat. No. 5,938,652 discloses an absorbent garment having a selectively elasticized waist flap that forms a pouch to contain the body exudates. The specific waist flap described therein is said to conform better to the wearer's back during use, while the remainder of the absorbent garment is positioned away from the wearer's back, thus forming a pouch.

U.S. Pat. No. 5,451,219 discloses a stretchable absorbent garment whereby the liquid permeable top sheet is connected to the liquid impermeable back sheet in discrete and non-discrete regions thereby forming a series of longitudinal pleats having channels disposed therein. The absorbent garment is described as being capable of absorbing a desirable amount of liquids, and at the same time capable of being elastically stretchable and contractible to better conform to the wearer's body during use.

U.S. Pat. No. 5,411,498 discloses a stretchable shaped absorbent garment containing at least two generally parallel elastomeric strands stretch bonded to the garment along its length. When the garment is in a relaxed state, the elastomeric strands are reduced in length rendering the garment stretchable.

U.S. Pat. No. 5,957,908 discloses an elastomeric ear panel used with “convertible” absorbent garments, whereby the ear panels (or flaps) are said to enable the use of the garment as a conventional diaper, or as a “pull-on” diaper. The specifically described ear panels impart the stretchability characteristics described therein. The elastomeric ear panels may be formed either from the top sheet or back sheet material, or may be separately formed and then affixed to the garment in the rear waist area.

U.S. Pat. No. 5,496,298 discloses an elastomeric ear suitable for use on an absorbent garment. The disclosed elastomeric ear has first and second connecting edges that are non-parallel to avoid undesirable red marking of the skin that occurs when the rear waist region of conventional absorbent garments are stretched and attached to the opposing waist region. The elastomeric ears are formed separately and attached to the absorbent garment during assembly.

U.S. Pat. No. 5,685,873 discloses differentially stretchable ears for use on an absorbent garment. The garment features childproof fastening in a disposable diaper.

U.S. Pat. No. 6,325,787 discloses an absorbent garment with an extensible waist formed using a structural elastic-like film that exhibits elastic-like behavior without the use of added elastics.

The disclosures of all of the aforementioned United States patents are incorporated by reference herein in their entirety, and in a manner consistent with this disclosure.

### SUMMARY OF THE INVENTION

It would be desirable to provide an absorbent garment with highly stretchable and contoured sides that, when fastened around the waist of the user, achieves a side panel contoured effect that shapes itself around the waist region of the user, maintains the fit of the garment despite user movement and increased weight due to collected body exudate, and does not produce stress marks (typically indicated by red markings) on the skin of the user. It also would be desirable to provide a garment with stretchable side panels that may be stretched to enable easy fastening, as for example a diaper that, once fastened, may be removed and replaced and maintain good fit without unfastening. It also would be desirable to provide a method of making a garment that has a top sheet, back sheet, and side panels that provides the aforementioned desirable features from a unitary composite sheet, with substantially no waste of the unitary composite sheet materials. Finally, it would be desirable to provide an absorbent garment, and a method of preparing an absorbent garment, with integral elastomeric side panels where the rear waist edge including the integral elastomeric side panels extends laterally in an unstretched condition at a distance greater than the lateral width of the unitary composite sheet from which the garment is continuously assembled, allowing the same garment to fit a variety of user sizes.

The invention is designed to provide these desirable features, plus other additional benefits. It is therefore a feature of the invention to provide a garment having a contoured body fit when placed on a user with highly stretchable contoured sides that maintains the fit of the garment despite user movement and increased weight due to collected body exudate yet does not produce stress marks on the skin of the user when the garment is fitted around the waist of the user. It is an additional feature of an embodiment of the invention to provide an absorbent garment that is capable of fitting a wider variety of user sizes, and can be removed and replaced while maintaining a good fit without unfastening the garment. It is yet another feature of an embodiment of the invention to provide an absorbent garment that includes integral elastomeric side panels that are formed from the unitary composite sheet used to form the garment, with substantially no waste of the unitary composite sheet. It is yet another feature of the invention to form a rear waist containment flap in the garment. Finally, it is yet another feature of the invention to provide a rear waist edge of an absorbent garment including integral elastomeric side panels that, when in the relaxed state, can extend laterally a distance greater than the lateral width of the unitary composite sheet from which the garment is formed.

In accordance with these and other features of various embodiments of the invention, there is provided an absorbent garment having a top sheet, a back sheet, and an absorbent core disposed between the top sheet and the back sheet. The top sheet and the back sheet form a first waist region, a second waist region longitudinally opposite the first waist region, and a crotch region between the first and second waist regions. A rear waist edge is formed from the first waist region and from integral elastomeric side panels laterally extending from the first waist region, where the integral elastomeric side panels each contain a fastening mechanism. The integral elastomeric side panels and the fastening mechanisms disposed thereto have a first waist edge and a crotch edge. The integral elastomeric side panels are formed from the unitary composite sheet from which the absorbent garment is continuously assembled and, when in the relaxed state, allow the rear waist edge to extend laterally a distance greater than the lateral width of the unitary composite sheet.

One or more target devices are disposed in the second waist region, whereby the fastening mechanisms disposed on the integral elastomeric side panels are capable of communicating or otherwise associating with the one or more target devices in the second waist region. The integral elastomeric side panels are made of a unidirectional or multidirectional elastomeric material.

In accordance with another feature of an embodiment of the invention, there is provided a method of making an absorbent garment that includes preparing a top sheet, a back sheet, and an absorbent core, and disposing the absorbent core between the top sheet and the back sheet. The top sheet, back sheet, and absorbent core are prepared and arranged such that the top sheet and the back sheet form a first waist region, a second waist region longitudinally opposite the first waist region, and a crotch region between the waist regions. The method also includes preparing a pair of integral elastomeric side panels from the same unitary composite sheet that extend laterally away from opposite sides of the first waist region, and in such a manner that assembly is simplified and waste of the unitary composite sheet material from which the absorbent garment is continuously assembled is substantially eliminated.

The method further includes disposing fastening mechanisms substantially on the lateral distal edges of the integral elastomeric side panels and disposing one or more target devices in the second waist region capable of communicating with the fastening mechanisms.

These and other features of the invention will be readily apparent from the Detailed Description that follows, along with reference to the drawings appended hereto.

### BRIEF DESCRIPTION OF THE DRAWINGS

FIG. 1 is a partially cut-away view of an embodiment of the invention, shown with elastic members fully stretched in the main portion of the garment in the laterally extending integral elastomeric side panels;

FIG. 2 is a cross sectional view of the embodiment of FIG. 1, as seen from reference line A-A, in a stretched condition;

FIG. 3 is a side view of the absorbent garment illustrated in FIG. 1 in use on a wearer;

FIG. 4 is a front view of the absorbent garment illustrated in FIG. 1 in use on a wearer;

FIG. 5 is a plan view of a unitary composite sheet formed to assemble the integral elastomeric side panels according to one embodiment of the invention;

FIG. 6 is a plan view of a unitary composite sheet formed to assemble the integral elastomeric side panels according to another embodiment of the invention;

FIG. 7 is a schematic illustrating one method to fold partially severed lateral side portions of the unitary composite sheet to form the integral elastomeric side panels according to one embodiment of the invention;

FIG. 8 is a schematic illustrating one method to fold partially severed lateral side portions of the unitary composite sheet to form the integral elastomeric side panels according to another embodiment of the invention;

FIG. 9 is a top view of the absorbent garment as prepared according to the schematics illustrated in FIGS. 7 and 8; and

FIG. 10 is a top view of the absorbent garment as prepared according to another embodiment of the invention.

### DETAILED DESCRIPTION OF THE PREFERRED EMBODIMENTS

As used herein, the terms “absorbent article,” “absorbent garment” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. The expression “unidirectional elastic” denotes an elastic material that is capable of being stretched either in the lateral or longitudinal direction. The expression “multidirectional elastic” denotes an elastic material that is capable of being simultaneously stretched in both the lateral and longitudinal directions.

The expression “unitary composite sheet” means a continuously supplied sheet of a composite of materials in a machine direction including the various layers that are assembled to form an absorbent garment. The various layers of the unitary composite sheet may comprise a back sheet and a top sheet, with an absorbent core disposed between the back sheet and the top sheet. Additional features of the absorbent garment may be included as part of the various layers of the unitary composite sheet such as fastening mechanisms, target devices, elastic elements or foam, leg gathers, standing leg gathers, or any other features that may be incorporated into an absorbent garment to provide desirable features for the garment. For example, the absorbent core may be wrapped in tissue, and may also include such features as fluid acquisition layers and transfer layers. A barrier layer may be disposed between the back sheet and the absorbent core. Other layers that may be included in the unitary composite sheet are well known to those skilled in the art.

The expressions “upper layer,” “lower layer,” “above” and “below,” which refer to the various components included in the absorbent core and other components of the invention (including the layers surrounding the absorbent core, if present) are used merely to describe the spatial relationship between the respective components. The upper layer or component “above” the other component need not always remain vertically above the core or component, and the lower layer or component “below” the other component need not always remain vertically below the core or component.

The term “component” can refer, but is not limited, to designated selected regions, such as edges, corners, sides or the like; structural members, such as elastic strips, absorbent pads, stretchable layers or panels, layers of material, or the like; or a graphic.

The term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed from or applied directly or indirectly to the surface of an element, formed from or applied between layers of a multiple layer element, formed from or applied to a substrate that is placed with or near the element, formed from or applied within a layer of the element or another substrate, or other variations or combinations thereof.

The terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

The invention relates to an absorbent garment that typically includes a top sheet, a back sheet, and an absorbent core disposed therebetween. The top sheet and back sheet may be coterminous with one another, or one may be larger and “overlay” the other. The top sheet and the back sheet form a first waist region, a second waist region longitudinally opposite the first waist region, and a crotch region between the first and second waist regions.

A rear waist edge is formed from the first waist region and from integral elastomeric side panels laterally extending from one or both lateral edges of the first waist region, where the integral elastomeric side panels each include a fastening mechanism. The integral elastomeric side panels and the fastening mechanisms disposed thereon have a first waist edge and a crotch edge. The integral elastomeric side panels are formed from the unitary composite sheet from which the absorbent garment is continuously assembled. The rear waist edge is capable, while in a relaxed condition, of extending in the lateral direction of the garment a greater distance than the lateral width of the unitary composite sheet.

One or more target devices may be disposed in the second waist region, whereby the fastening mechanisms disposed on the integral elastomeric side panels are capable of communicating or otherwise associating with the one or more target devices in the second waist region. The integral elastomeric side panels are made of a unidirectional or a multidirectional elastomeric material, preferably an elastomeric material that is stretchable in the lateral direction. While not intending on being bound by any theory of operation, it is believed that the use of unidirectional or multidirectional elastomeric materials enables the use of lesser amounts of material for the integral elastomeric side panels.

In one preferred embodiment of the invention, the fastening mechanism may be a hook portion of a hook and loop fastener, or an integral part of the integral elastomeric side panel or an outer layer of the garment. The one or more target devices may be the loop portion of a hook and loop fastener, or may be an integral part of the back sheet or other outer layer of the garment. In another embodiment, the fastener may be an adhesive tape and the target devices may comprise a tape receiving surface.

The invention now will be described with reference to the attached drawings illustrating preferred embodiments of the invention. For clarity, features that appear in more than one Figure have the same reference number in each Figure.

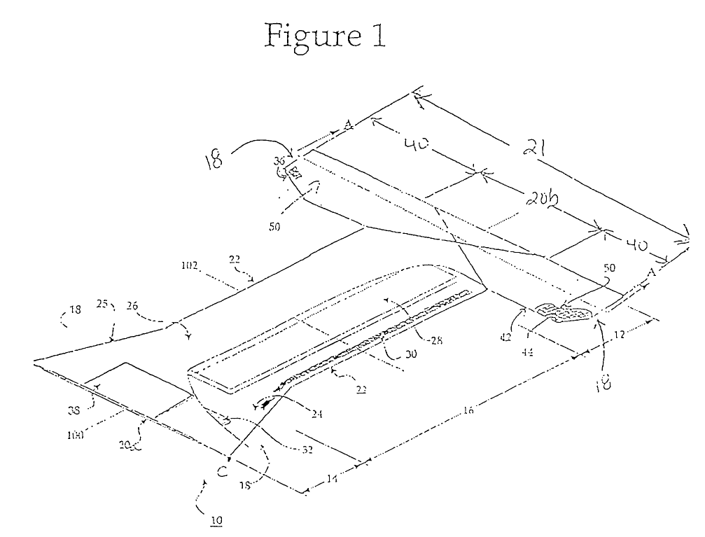

FIG. 1 is a partially cut away depiction of an exemplary embodiment of a garment 10 of the invention. The embodiment shown in FIG. 1 is an infant's diaper, however, this depiction is not intended to limit the invention. The garment 10 of FIG. 1 is depicted in a generally flattened position, with the body-facing side facing down, and with the various elastic components depicted in their extended position for clarity. In the flattened position, the garment 10 may have a generally T-shaped structure, but it may also have any other shape suitable for the given application, such as a rectangular shape, a trapezoidal shape, and the like. The various components of the garment 10 are typically continuously supplied in a machine direction to form the absorbent garment, according to techniques well known to those skilled in the art. The composite of these continuously supplied components that is prepared during assembly of the absorbent garment comprise what is herein referred to as the unitary composite sheet.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region, the waist encircling region being defined by front longitudinally distal edge 20a and rear waist edge 21. Front longitudinally distal edge 20a comprises the distal edge of front waist region 14 as seen in FIG. 1 between Points B and C. Rear waist edge 21 is formed from rear longitudinally distal edge 20b and first waist edges 40 of integral elastomeric side panels 50. The waist-encircling region may comprise a first waist region 12 disposed adjacent to, for example, the back waist region of a wearer's body, and a second waist region 14 disposed adjacent to, for example, the front waist region of a wearer's body. The first and second waist regions 12, 14, may correspond to the back and front of the wearer's body, respectively, depending on whether garment 10 is attached in front of or behind the subject wearer. The first and second waist regions are joined together at or near their lateral edges 18, causing front longitudinally distal edge 20a and rear waist edge 21 of the garment 10 to form the perimeter of a waist opening. Preferably, these edges overlap. A crotch region 16 extends between the first and second waist regions 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. Alternatively, the top sheet 24 may define an area that is not coterminous with the back sheet 26. When the garment 10 is being worn, the top sheet 24 faces the wearer's body, and the back sheet 26 faces away from the wearer. An absorbent core 28 preferably is disposed between at least a portion of the top sheet 24 and the back sheet 26.

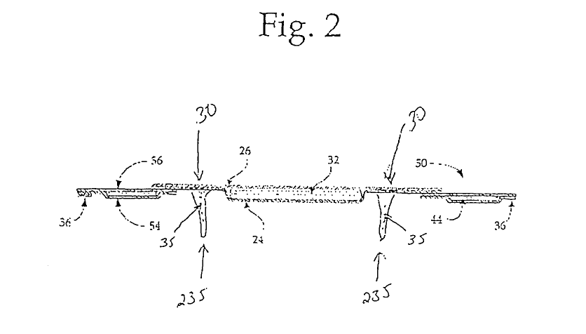

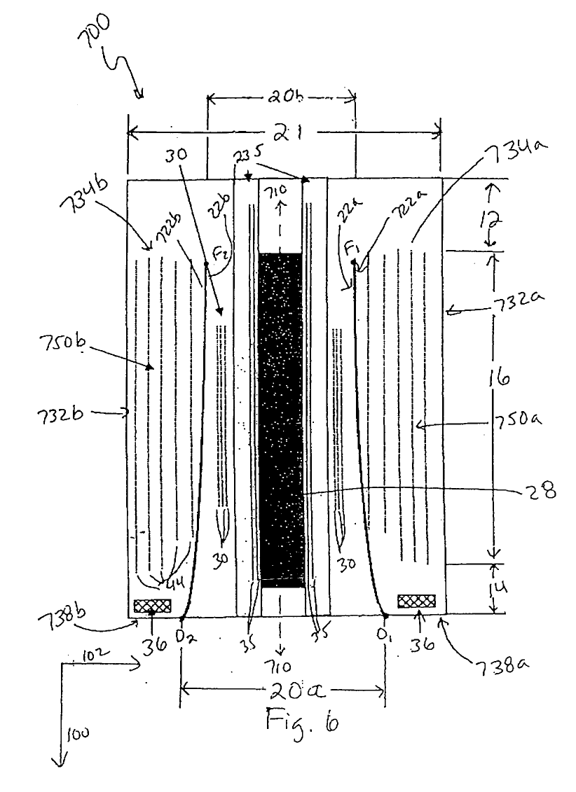

The garment 10 also includes a pair of laterally opposed integral elastomeric side panels 50 in the first waist region 12 that may be formed from partially severed lateral side portions formed from the unitary composite sheet during the assembly process. Integral elastomeric side panels 50 include a first waist edge 40, and a longitudinally opposed crotch edge 42. It is preferred that integral elastomeric side panels 50 include unidirectional or multidirectional elastic materials 44 disposed between upper 54 and lower 56 materials used to make integral elastomeric side panels 50, as shown in FIG. 2. As will be discussed in reference to FIGS. 5 through 10, the lower material 56 will preferably be made of the same material used to make the back sheet 26, and may be treated in the same fashion so as to render it hydrophobic, and/or fluid impervious. Similarly, the upper material 54 will preferably be comprised of the same material used to make top sheet 24, and may be treated in the same fashion so as to render it hydrophilic, and/or fluid pervious. Integral elastomeric side panels 50 may also be formed from one or more of the other layers of the unitary composite sheet, as for example the top sheet material or the back sheet material.

An embodiment of the invention may comprise various additional features. One or more pairs of elastic gathers 30 may extend adjacent the crotch edges 22. The garment 10 may also comprise one or more waste containment systems, such as inboard standing leg gathers (as shown in FIGS. 2, 5, 6, 8, and 10). One or both of the first and second waist regions 12, 14 may also be equipped with strips of elastic waist foam 32 or other elastically extensible material, which help contract the garment around the wearer's waist, providing improved fit and leakage prevention.

A pair of tabs (not shown) may optionally extend laterally away from opposite lateral edges 18 of the integral elastomeric side panels 50 of the garment 10. The tabs may comprise an elastically extensible material, designed to stretch around a wearer's waist to provide improved fit, comfort, and leakage protection. Such elasticized tabs may be used in conjunction with, or in lieu of, waist foam 32, or other elastically extensible materials 32. Alternatively, an elastic material may be disposed between certain layers of the unitary composite sheet, or the tabs may not be stretchable. In a preferred embodiment, though, tabs will not be included in the garment 10.

At least one fastening mechanism 36 (collectively referred to as “fasteners 36”) preferably is disposed on each opposite lateral edge 18 of integral elastomeric side panels 50 for attaching the integral elastomeric side panel 50 to the second waist region 14, thereby providing the garment 10 with a pant-like shape, and enabling garment 10 to be fixed or otherwise fitted on the wearer. The fasteners 36 may attach to one or more target devices 38 located in the second waist region 14. When tabs are disposed on integral elastomeric side panels 50, fastening mechanisms 36 may be disposed on each lateral edge of the tabs.

Although not shown in the drawings, the absorbent garment 10 may also include grips attached along one of its edges proximal to each opposite lateral edge 18 of integral elastomeric side panels 50 or tabs to enable a caregiver to pull the grips, and not on the ends of the integral elastomeric side panels or tabs, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

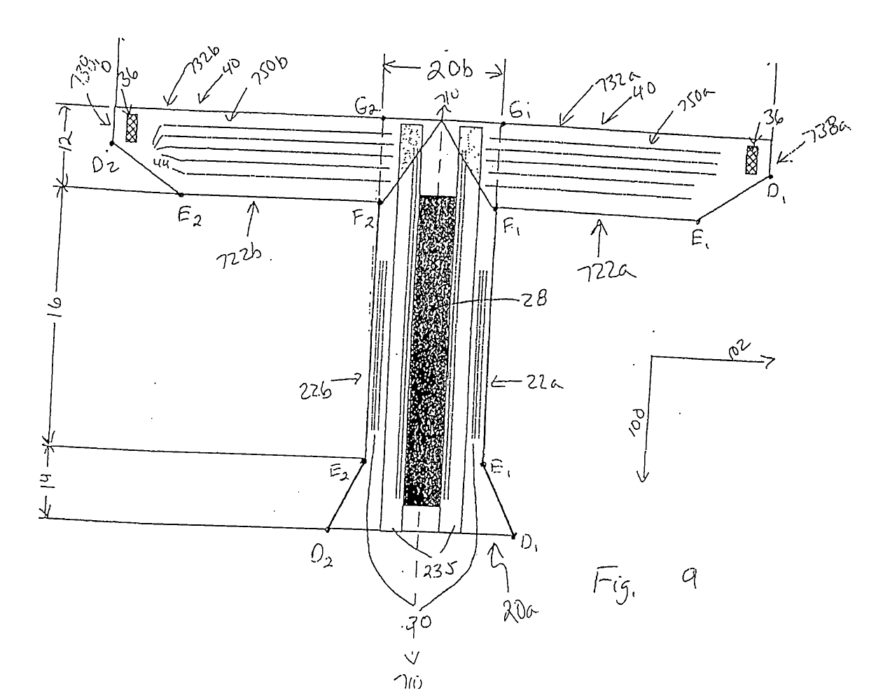

The top sheet 24, back sheet 26, upper material 54 of integral elastomeric side panel 50, and lower material 56 of integral elastomeric side panel 50 may be constructed from a wide variety of materials known in the art. The invention is not intended to be limited to any specific materials for these components. The top sheet 24, back sheet 26, upper material 54 of integral elastomeric side panel 50, and lower material 56 of integral elastomeric side panel 50 can be shaped and sized according to the requirements of each of the various types of absorbent garment, or to accommodate various user sizes. In an embodiment of the invention in which the garment 10 is a diaper or an adult incontinence brief, the garment 10 may have a T-shape, as seen in FIG. 1, or may have a rectangular, trapezoidal, hourglass shape, or other shape as seen in FIGS. 8 and 10, with the elastics shown in a stretched condition.

The back sheet 26 and lower material 56 of integral elastomeric side panel 50 (hereinafter referred to simply as “back sheet”) generally are made of any suitable pliable liquid impervious material known in the art or later discovered. Typical back sheet materials include films of polyethylene, polypropylene, polyester, nylon, and polyvinyl chloride and blends of these materials. For example, the back sheet 26 may be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably should have sufficient liquid imperviousness to prevent any leakage of fluids through the back sheet 26. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material.

The back sheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with the present application and invention. Materials for such a fibrous outer liner include a spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers. Either the back sheet 26 or covering fabric may have imprinted thereon a characteristic mark, graphic, or illustration.

The top sheet 24 and upper material 54 of integral elastomeric side panel 50 (hereinafter referred to simply as “top sheet”) may be made of any suitable relatively liquid-pervious material currently known in the art or later discovered that permits passage of a liquid therethrough. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The top sheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The invention is not intended to be limited to any particular material for the top sheet 24, and other top sheet materials will be readily apparent to those skilled in the art.

The top sheet 24 may further comprise several regions having different properties. In one embodiment of the invention, the laterally distal portions of the top sheet 24, especially those used to make integral elastomeric side panels 50 (e.g., upper material 54) are preferably substantially fluid impervious and hydrophobic, while the remainder of the top sheet 24 is hydrophilic and fluid pervious. Different top sheet properties, such as fluid perviousness and hydrophobicity, may be imparted upon all or just specific targeted areas of the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28 and form upper material 54 of integral elastomeric side panel 50, and the back sheet 26 is large enough to prevent leakage from the garment 10 and form lower material 56 of integral elastomeric side panel 50. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the top sheet 24 to the back sheet 26. The particular joining method may be dictated by the types of materials selected for the top sheet 24 and back sheet 26.

An absorbent core 28 preferably is disposed between the top sheet 24 and the back sheet 26 in at least the crotch region 16. The absorbent core 28 may extend into either or both of the first and second waist regions 12, 14. Although the absorbent core 28 depicted in FIG. 1 has a substantially rectangular shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10 by any method known in the art, in order to fix the absorbent core 28 in place.

The absorbent core 28 may be made from any suitable material or materials known in the art that are capable of absorbing or retaining liquids (e.g., urine and/or menses). Examples of suitable materials for use as the absorbent core 28 include creped cellulose wadding, absorbent foams, absorbent sponges, super absorbent polymers, absorbent gelling materials, fiberized cellulose, chemically stiffened, modified or cross-linked cellulose fibers, tow fibers, fluff pulp having tissue or synthetic materials between the absorbent core 28 and the top sheet 24, comminuted wood pulp (commonly known as “airfelt”) or any equivalent material or combination of materials. The size and capacity of the absorbent material should correspond to the application, for example, an incontinent brief for an adult may require a larger absorbent core than a diaper for a child. Zoned absorbency may also be used, if desired. For example, more absorbent capacity may be located in particular regions of the garment 10 depending on the gender of the intended wearer. The invention is not intended to be limited to any specific materials or sizes for use in the absorbent core 28.

In a preferred embodiment, the absorbent core 28 comprises super absorbent polymer distributed within a fibrous structure. Absorbent cores of this type are known in the art, and exemplary absorbent cores are described in U.S. Pat. Nos. 5,281,207, and 6,068,620, issued to Chmielewski et al., and U.S. Pat. No. 5,863,288, issued to Baker, the disclosures of each of which are herein incorporated by reference in their entirety.

Additional sublayers, transfer layers, acquisition layers, tissue wraps, and the like also may be incorporated into the absorbent core 28. Such layers may be provided to assist with transferring fluids to the absorbent core 28, handling fluid surges, preventing rewet, containing absorbent material, improving core stability, or for other purposes. For example, a substantially rectangular, preferably nonwoven, sublayer (not shown), having a basis weight of about 0.1-2 oz., preferably about 0.4-0.6 oz., may overlay absorbent core 28. Those skilled in the art are capable of selecting materials, dimensions, and locations for such layers without undue experimentation.

In a preferred embodiment, the absorbent core 28 is a laminate comprised of a layer of superabsorbent polymer material in the form of particles disposed between two air-laid tissues, first and second tissue layers (or “upper” and “lower” tissue layers). The first and second tissue layers contain the superabsorbent polymer material, improve lateral wicking of the absorbed exudates throughout the absorbent core 28 and provide a degree of absorbency.

The crotch region 16, or other regions of the garment 10, may also include mechanical sealing devices to provide the garment 10 with a leak-proof fit around the wearer. In a preferred embodiment, the crotch edges 22 each have one or more elastic leg gathers 30 placed along them to help the crotch edges 22 contract about the contours of the wearer's body, thereby providing a leak-resistant seal. Such elastic leg gathers 30 are known in the art, and are disclosed, for example, in U.S. Pat. No. 5,830,203, issued to Suzuki et al., the disclosure of which is herein incorporated by reference in its entirety in a manner consistent with the invention. The elastic leg gathers 30 may extend into the first waist region 12, and may extend as far as the front longitudinally distal edge 20a.

In another preferred embodiment, standing leg gathers (as shown in FIGS. 2, 5, 6, 9, and 10) are disposed on the top sheet 24. Standing leg gathers are strips of material that rise vertically from the surface of the top sheet 24 to provide additional sealing to the garment 10. The standing leg gathers may extend across all or part of the garment 10 along its longitudinal axis 100. Typically, one or more standing leg gather(s) is located laterally on either side of the absorbent core 28. The standing leg gathers may be made from folded portions of the top sheet 24 or back sheet 26, or may be made from additional strips of material. Each standing leg gather may be equipped with one or more elastic elements to help seal the gather to the wearer's body. Standing leg gathers are known in the art, and disclosed in U.S. Pat. No. 5,292,316, issued to Suzuki, the disclosure of which is herein incorporated by reference in its entirety in a manner consistent with the invention.

Any suitable elastic material may be used for the elastic leg gathers 30 and the standing leg gathers. Preferably, the elastic material can be stretched to between at least 10% to 300% of its original length without losing its resilience. The elastic material used for the elastic leg gathers 30 and standing leg gathers may, for example, comprise an elastic film, a multidirectional elastic aggregate such as elastic webbing, netting, or scrim elastic, or strands or bands of suitable elastic materials, such as natural or synthetic rubber, urethane elastomers, spandex, LYCRA and elastic polymers. Other suitable elastics will be apparent to those skilled in the art in light of the present teachings. The elastics materials may be attached to the garment 10 in any of several ways known in the art. For example, the elastic materials may be ultrasonically bonded, heat/pressure sealed using a variety of bonding patterns, or glued to the diaper 10 using a variety of adhesives. Other performance enhancing devices, such as pockets, baffles, and openings in the top sheet also may be used with the invention.

Any suitable unidirectional or multidirectional elastic material may be used as elastic material 44 in integral elastomeric side panel 50. Unidirectional elastic materials are capable of being stretched in one direction; multidirectional elastic materials are capable of being stretched in more than one direction. Elastic materials may include elastic webbing, netting, or scrim elastic, or strands or bands of suitable elastic materials that are disposed in either the lateral direction (to form unidirectional elastic material) or longitudinal and lateral directions (to form multidirectional elastic material), such as natural or synthetic rubber, urethane elastomers, spandex, LYCRA and elastic polymers. A particularly preferred elastic material for use in the invention is strands or bands of elastic elements disposed in the lateral dimension of the garment 10.

The elasticity of the integral elastomeric side panel 50 may be varied according to the type of elastic material used. In one embodiment, the elasticity of integral elastomeric side panel 50 may be varied by the number of elastic strands used. Preferably, about 2 to about 20 elastic strands are incorporated in the integral elastomeric side panels 50 in the substantially lateral dimension of the garment 10. The elastic strands may further be adjusted to provide varying degrees of elasticity in the integral elastomeric side panels 50 by associating the elastic strands with the integral elastomeric side panels 50 only in predetermined positions such that regions of the integral elastomeric side panels 50 will shirr, or contract, where the elastic strands are associated, and will not shirr, or contract, in those areas where elastic strands have not been associated. Those skilled in the art are capable of providing a unidirectional or multidirectional elastic material 44 in integral elastomeric side panel 50 with varying degrees of elasticity in the lateral dimension of the garment 10 using the guidelines provided herein.

The first and second waist regions 12, 14 may be joined to one another by fastener 36, or a plurality of fasteners 36, each of which may be disposed substantially on a lateral side edge 18 of integral elastomeric side panel 50 or a lateral edge of a tab, if included. The fasteners 36 may interact with one or more target devices 38 disposed in the second waist region 14 to provide sufficient gripping force to hold the garment 10 on a wearer. While the embodiment depicted in FIG. 1 shows the fasteners 36 being located in the first waist region 12, and the target device 38 being in the second waist region 14, skilled artisans will recognize that these locations may be reversed. Exemplary embodiments of the fasteners 36 and target devices 38 are provided herein, however, the invention is not intended to be limited to any particular construction or materials for these parts.

The integral elastomeric side panels 50, fasteners 36, and target devices 38 preferably are located and sized so that they may be used to wrap the first and second waist regions 12, 14 around wearers having a variety of sizes. For example, additional elastics (not shown) may be added to the garment 10 in one or both of the first and second waist regions 12, 14 in order to increase the range of user sizes that may be fitted by a single size garment 10. In a preferred embodiment, a strip of elastic foam 32 is located in each waist region to provide the waist regions with elasticity, additional comfort, and leakage prevention. The strips of elastic foam 32, such as an elastic polyurethane foam, also may help front longitudinally distal edge 20a and rear waist edge 21 to resist doubling over (“rolling”) during use.

When included, tabs preferably are attached to the garment 10 at opposite lateral edges 18 of the integral elastomeric side panels 50, and extend distally away from the center of the garment 10. The tabs may be attached to the integral elastomeric side panels 50 using any method that provides a suitably strong bond. They may be attached in an edge-to-edge fashion with edges 18 of integral elastomeric side panel 50, or may overlap the top sheet 24, back sheet 26, upper material 54 of integral elastomeric side panel 50, lower material 56 of integral elastomeric side panel 50, or another part of the garment 10, in order to provide a stronger or larger bond area. Exemplary methods for attaching the tabs to the garment 10 include: ultrasonic welding, thermal bonding, adhesive bonding, stitching, stapling, combinations of the foregoing, and the like. A skilled artisan will be able to attach the tabs to the garment 10 without undue experimentation.

The tabs may comprise any suitable material. Nonwoven materials, such as those described elsewhere herein, may be particularly suitable for making the tabs. The tabs comprise an elasticized material, such as a laminate of nonwoven sheets, and an elastic material 46. Also in a preferred embodiment, the tabs are comprised of a nonelastic material that is easily bonded to the garment 10 and to the fasteners 36 by any of various means known in the art.

The fasteners 36 may comprise an adhesive tape, a portion of a mechanically interlocking fastener, such as the hook or loop portion of a hook and loop fastener, or a button, snap, or any other suitable fastening mechanism known in the art. In one embodiment, fasteners 36 may be integral to a component of the garment 10, such as the back sheet 26 or other outer layer. Preferably, the fasteners 36 may be sealed and released numerous times without losing their holding and/or fastening ability. The at least one target device 38 may be associated with a surface of the garment 10, or it may be an integral part of the surface of the garment. That is, the surface of the garment may itself act as a target device that will suitably interact with the fasteners 36. The selection and manufacture of fasteners 36 and target devices 38 is known in the art, and a skilled artisan will be able to implement such structures and devices without undue experimentation.

In a preferred embodiment, the fasteners 36 is the hook portion of a hook and loop fastener. The fasteners 36 may comprise a separate strip of material having a backing sheet with the hooks protruding therefrom, or the fasteners 36 may protrude directly from the surface of each integral elastomeric side panel 50. The fasteners 36 also may protrude beyond the opposite lateral edges 18 of integral elastomeric side panels 50. In one embodiment, the fasteners 36 may be a hook formed as an integral component of the back sheet 26 or other outer layer of the garment 10. The design and construction of such fasteners 36 is disclosed, for example, in U.S. Pat. No. 5,624,429 to Long et al., the disclosure of which is incorporated herein by reference in its entirety and in a manner consistent with the invention.

Also in a preferred embodiment, the at least one target device 38 is the loop portion of a hook and loop fastener that is selected to mechanically engage with the fasteners 36 to provide a secure, yet releasable and resealable bond. The target device 38 may be provided as a separate strip of material having a backing sheet with the loops protruding therefrom. In such a case, the target device or devices 38 may be attached to the outermost layer of the garment by any suitable means. In another embodiment, the target device 38 may also comprise a portion of the outer surface material of the garment (whether the outer surface is the back sheet 26 or an outer cover) that can mechanically engage with the fasteners 36, or that is mechanically or chemically treated to be able to mechanically engage with the fasteners 36.

In any case in which the fasteners 36 or target device 38 may cause irritation of the wearer's skin, it is preferred that such fasteners 36 or target devices 38 be shielded from contact with the wearer, such as by covering the target or fasteners 38, 36 with a protective cover prior to being fastened together. Such a protective cover may also serve to prevent the target devices or fasteners from becoming contaminated and inoperable.

FIG. 2 is a cross sectional view of a portion of the embodiment shown in FIG. 1, as seen from reference line A-A. FIG. 2 shows elastic material 44 in integral elastomeric side panels 50. Those skilled in the art will appreciate that integral elastomeric side panels 50 may contain unidirectional or multidirectional elastic material.

As shown in FIG. 2, lower material 56 and upper material 54 of integral elastomeric side panel 50 surround and otherwise envelop unidirectional or multidirectional elastic material 44. Lower material 56 and upper material 54 of integral elastomeric side panel 50 are preferably made of the same material as back sheet 26 and top sheet 24, respectively, or they may be made of other materials. For example, in preparing the unitary composite sheet, a combination of materials may be disposed or situated along with the back sheet material and top sheet material such that, upon partial severance of lateral side portions (discussed in reference to FIGS. 5 through 10), integral elastomeric side panels 50 comprise a material different from the back sheet 26 or top sheet 24. Other layers also may be present between lower material 56 and unidirectional or multidirectional elastic 44, and/or upper material 54 and unidirectional or multidirectional elastic 44. In addition, other layers may be disposed outside lower material 56 and/or upper material 54 of integral elastomeric side panel 50. In one embodiment, fasteners 36 are disposed on the opposite lateral edges of integral elastomeric side panels 50.

Additional features such as leg gather elastics 30, standing leg gathers 235, and standing leg gather elastics 35 are also shown. FIG. 2 also depicts an embodiment wherein waist foam element 32 is present in the first waist region 12 [FIG. 1], but skilled artisans will appreciate that first waist region 12 [FIG. 1] need not contain any elastic element, or it may contain elastic materials other than foam.

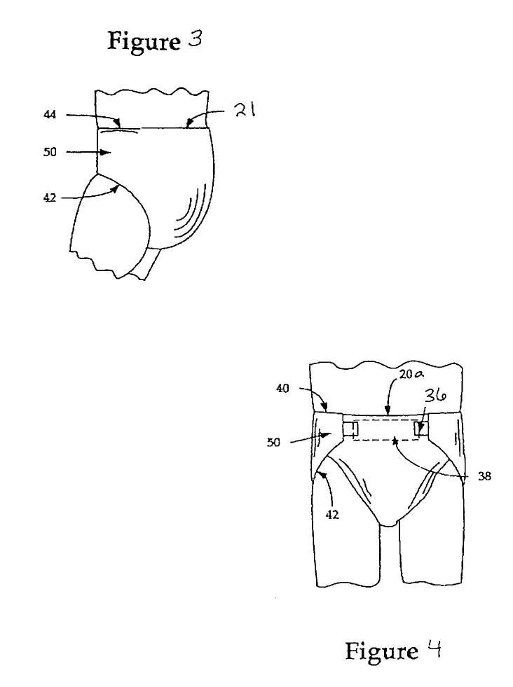

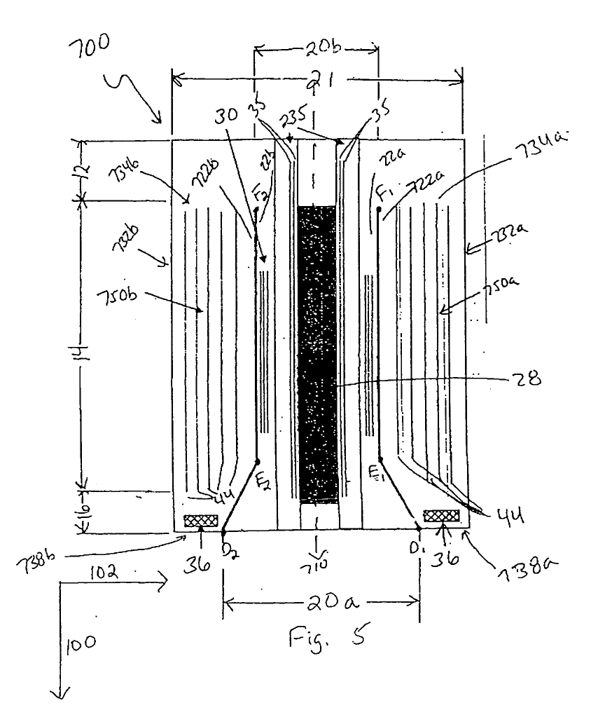

FIGS. 3 and 4 illustrate a preferred absorbent garment 10 as it is fitted in on a wearer. As shown, crotch edge 42 of integral elastomeric side panel 50 is contoured after the garment is stretched and fitted on a wearer. In addition, first waist edge 40 and crotch edge 42 of integral elastomeric side panel 50 may not be substantially parallel to one another when the garment is fitted on a wearer.

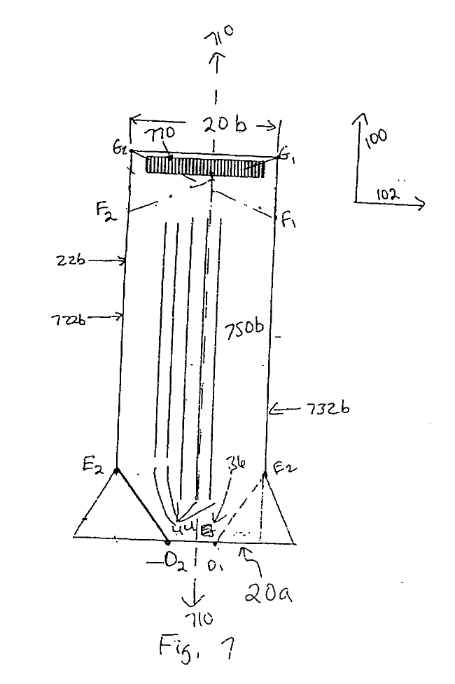

Alternative methods of forming integral elastomeric side panels 50 from a unitary composite sheet are illustrated in FIGS. 5-10. FIGS. 5 and 6 depict preferred embodiments of forming integral elastomeric side panels 50 according to the invention from the unitary composite sheet. In FIGS. 5 and 6, the unitary composite sheet 700 has a lateral dimension 102 and a longitudinal dimension 100 which substantially correspond to the lateral and longitudinal dimensions of the finished absorbent garment. Lateral side portions 750a, b are formed when the unitary composite sheet 700 is severed along lines in the substantially longitudinal dimension defined between predetermined Points D₁ through F_{1 and D}₂ through F₂. Each lateral side portion 750a, b has an inner distal edge 722a, b, and an outer distal edge 732a, b as well as a rear longitudinal edge 734a, b and a front longitudinal edge 738a, b. As the unitary composite sheet 700 advances on an assembly line in a machine direction 100, it can be partially severed in any manner now known or later developed on each lateral side in the substantially longitudinal dimension beginning at Point D and following a predetermined route to Point F. In the embodiments illustrated, lateral side portions 750a, b are mirror images of each other, although this is not a vital feature to the invention and is not intended to limit it as such. Alternatively, lateral side portions 750a, b may be formed by any method known or later developed in the art, for example by causing a series of perforations along a line defined by Points D through F and tearing the unitary composite sheet or by directing a laser along a line defined by Points D through F to form the lateral side portions 750a, b. Note that, in the embodiment depicted in FIG. 5, points D₁ and D₂ correspond substantially to Points B and C in FIG. 1. In a preferred embodiment, rear longitudinal edge 734a, b is longer in the lateral dimension of the unitary composite sheet 700 than front longitudinal edge 738a, b.

Elastic elements 30, 35, and 44 can be disposed between and associated with different layers of the unitary composite sheet 700 during assembly of the absorbent garment 10. They may be disposed before or after the unitary composite sheet 700 is severed to form lateral side portions 750a, b. In the depicted embodiments of FIGS. 5 and 6, leg elastics 30, standing leg gather elastics 35, and integral elastomeric side panel elastics 44 may be disposed between and associated with various layers of the unitary composite sheet 700. This depiction is not intended to limit the invention, as the elastic elements may be disposed as desired according to techniques well known in the art.

The elasticity of the leg gathers, standing leg gathers, and integral elastomeric side panels 50 may be varied according to the number, elasticity, and degree of association of the elastic elements 30, 35, and 44, respectively. In one embodiment, about 2 to about 20 elastic elements 44 are disposed in the substantially longitudinal direction 100 within each of lateral side portions 750a, b. Elastic elements 44 are stretched prior to association. In a preferred embodiment, elastic elements 44 are associated using adhesive in substantially the middle of lateral side portions 750a, b in the longitudinal direction 100, with substantially no association of the ends of the elastic elements to the lateral side portions 750a, b. After association, the tension on the elastic elements is released and they contract, causing the portions of lateral side portions 750a, b where elastic elements 44 have been associated shirr, or gather. In contrast, those portions of lateral side portions 750a, b where elastic elements 44 have not been associated do not shirr, or contract. In this manner, the elasticity of lateral side portions 750a, b can be varied, which can be adjusted to achieve the desired fit of the garment in the waist region to a variety of users having different waist sizes. Similar adjustments can be made to the leg gather elastics 30 and standing leg gather elastics 35, as known to those skilled in the art.

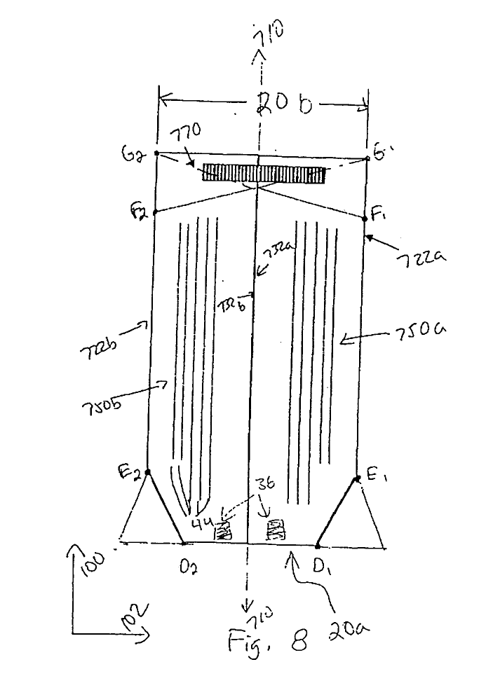

FIG. 7 depicts one method of folding lateral side portions 750a, b to form integral elastomeric side panels 50. First, each lateral side portion 750a, b is folded across the center line 710 of unitary composite sheet 700 in a substantially lateral direction such that inner distal edges 722a, b of lateral side portions 750a, b overlay and are substantially coterminous with crotch edges 22a, b, and outer distal edges 732a, b overlay and are substantially coterminous with opposite inner distal edges. Specifically, lateral side portion 750a is folded such that inner distal edge 722a overlays and is substantially coterminous with crotch edge 22a and outer distal edge 732a overlays and is substantially coterminous with opposite inner distal edge 722b and crotch edge 22b. Lateral side portion 750b is folded such that inner distal edge 722b overlays and is substantially coterminous with crotch edge 22b and outer distal edge 732b overlays and is substantially coterminous with crotch edge 22a and crotch edge 22a. A bond 770 may be created between the layers thus formed at rear longitudinally distal edge 20b by any known or later developed bonding technique.

FIG. 8 illustrates another folding option to form lateral side portions 750a, b from unitary composite sheet 700 that may be utilized to form integral elastomeric side panels 50, according to the embodiments shown in FIGS. 5 and 6. In FIG. 8, inner distal edges 722a, b of lateral side portions 750a, b are folded partially toward the center line 710 such that outer distal edges 732a, b meet at the centerline 710 of absorbent garment 10 although outer distal edges 732a, b may overlap as well.

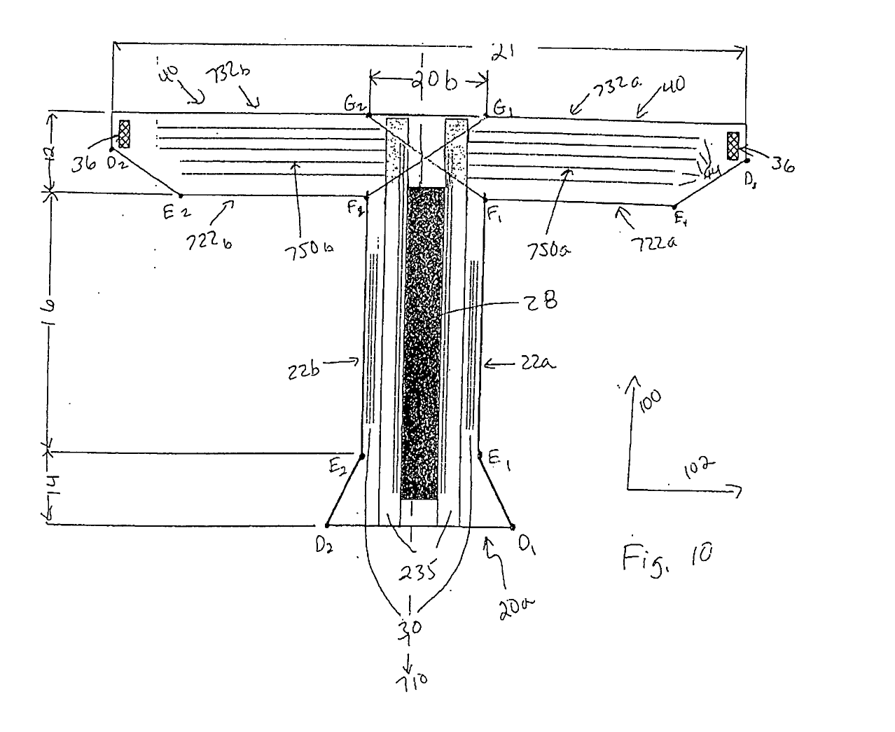

As seen in FIG. 9, each lateral side portion 750a, b then may be folded by bringing each front longitudinal edge 738a, b toward rear longitudinally distal edge 20b along fold lines F₁ to G₂ and F₂ to G₁. Upon folding, outer distal edges 732a, b of lateral side portions 750a, b are substantially coterminous with rear longitudinally distal edge 20b. Specifically, outer distal edge 732a is folded such that Point D₁ defines the right lateral limit of rear waist edge 21, and lateral edge 732b is folded such that Point D₂ defines the left lateral limit of rear waist edge 21.

Outer distal edges 732a, b correspond to the first waist edge 40 of integral elastomeric side panels 50 (FIG. 1), which themselves correspond to lateral side portions 750a, b. Thus, rear waist edge 21 includes both the rear longitudinally distal edge 20b (which has a length substantially the same as the distance between Points G₁ and G₂) and the first waist edges 40 (e.g., outer distal edges 732a, b) of lateral side portions 750a, b, which defines the lateral limits of rear waist edge 21, as denoted by points D₁ and D₂. The distance between Points D₁ and D₂, or the lateral width of the rear waist edge 21 is greater than the width of the rear longitudinally distal edge 20b, and is also greater than the lateral width of unitary composite sheet 700 between lateral edges 732a and 732b shown in FIGS. 5 and 6.

Features such as fasteners 36 can be disposed on the lateral side portions 750a, b in such a predetermined position that they are appropriately positioned in the final absorbent garment to mate with target devices 38.

Although the description of the exemplary embodiments shown in FIGS. 5 through 9 depict integral elastomeric side panels 50 as formed by severing the fully assembled unitary composite sheet 700, this is not intended to limit the invention. Rather, integral elastomeric side panels 50 may be severed from any combination of the layers of the unitary composite sheet. In addition, integral elastomeric side panels 50 may be folded at any point during assembly of the absorbent garment. For example, integral elastomeric side panels 50 may be severed from the back sheet material, and may be folded to form the rear waist edge 21 before other layers are associated with the back sheet material.

FIG. 10 illustrates another embodiment for folding lateral side portions 750a, b to form integral elastomeric side panels 50. Lateral side portions 750a, b may be crisscrossed in the depicted folding pattern, and may require additional cuts in the lateral side portions 750a, b to permit them to cross each other in the first waist region. Those skilled in the art will be able to form integral elastomeric side panels 50 in varying combinations of the unitary composite sheet, without undue experimentation.

One advantage to the invention that is apparent to those skilled in the art is that integral elastomeric side panels can be formed during the same assembly process as the rest of the chassis of the absorbent garment, thus simplifying the assembly process. In addition, the design of the integral elastomeric side panels from the same unitary composite sheet that forms the chassis of the absorbent garment reduces the need for adhesive or other association techniques to associate the integral elastomeric side panels with the chassis; forms a much stronger associative bond between the integral elastomeric side panels and the chassis of the absorbent garment that will virtually not break due to association failures; and reduces the assembly process by at least one step that previously required that the integral elastomeric side panels be disposed on and associated with the chassis of the absorbent garment. In addition, a back pocket may be formed in the rear waist region that assists in the containment of body exudates in the absorbent garment. Finally, since the rear waist edge may extend laterally in the absorbent garment a greater length than the width of the unitary composite sheet, the garment can be adjusted to comfortably accommodate a wider variety of wearer sizes.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

1. An absorbent garment having a longitudinal dimension and a lateral dimension comprising:
a top sheet;
a back sheet, whereby the top sheet and the back sheet form a first waist region, a second waist region longitudinally opposite the first waist region, and a crotch region therebetween;
an absorbent core disposed between the top sheet and the back sheet;
at least one side panel, wherein the at least one side panel laterally extends from at least one lateral edge of the first waist region, wherein further the at least one side panel and first waist region comprises a rear waist edge;
at least one fastening mechanism disposed substantially on a lateral edge of the at least one side panel; and
one or more target devices disposed in the second waist region, where the at least one fastening mechanism and the one or more target devices are capable of attaching to one another, the one or more target devices being located so that the first waist region and second waist region of the garment may be joined to one another to secure the garment on a wearer,
whereby the absorbent garment is formed in a continuous assembly process from a composite sheet comprising layered sheets of top sheet material and back sheet material, the composite sheet being continuously supplied in the longitudinal dimension and having a maximum width in the lateral dimension, and wherein the at least one side panel comprises at least one of the layered sheets of the composite sheet, and whereby further the lateral dimension of the rear waist edge while in a relaxed state is capable of extending laterally a length greater than the maximum width of the composite sheet in the lateral dimension.

2. The absorbent garment of claim 1, wherein the at least one side panel comprises an elastomeric material.

3. The absorbent garment of claim 2, wherein the elastomeric material comprises elastic webbing, netting, scrim elastic, or strands or bands of elastic materials that are disposed in the longitudinal, lateral, or longitudinal and lateral dimensions of the at least one side panel, or combinations thereof.

4. The absorbent garment of claim 3, wherein the strands or bands of elastic materials comprise natural or synthetic rubber, urethane elastomers, spandex, LYCRA, elastic polymers, and mixtures thereof.

5. The absorbent garment of claim 3, wherein the elastomeric material comprises about 2 to about 20 strands or bands of elastic materials disposed on the at least one side panel.

6. The absorbent garment of claim 5, wherein the strands or bands of elastic materials are partially associated with the at least one side panel such that the elasticity of the at least one side panel is varied across its lateral dimension.

7. The absorbent garment of claim 1, wherein the at least one fastening element comprises a hook portion of a hook and loop fastener and the one or more target devices comprise the loop portion of a hook and loop fastener.

8. The absorbent garment of claim 7, wherein at least one of the hook or loop portion are integrally formed from a layer of the composite sheet.

9. The absorbent garment of claim 1, wherein the at least one fastening element comprises an adhesive tape and the one or more target devices comprises a tape receiving surface.

10. The absorbent garment of claim 1, wherein the at least one side panel comprises two separate sheets.

11. The absorbent garment of claim 1, wherein the at least one side panel comprises a first material, a second material, and an elastic material, whereby the elastic material is disposed between the first material and the second material.

12. The absorbent garment of claim 11, wherein the first material comprises the back sheet material, and the second material comprises the top sheet material.

13. The absorbent garment of claim 1, wherein the first waist edge and crotch edge of the at least one side panel are substantially nonparallel.

14. The absorbent garment of claim 1, wherein the first waist edge and crotch edge of the at least one side panel are substantially parallel.

15. A method of making an absorbent garment comprising:
preparing at least a top sheet, a back sheet, and an absorbent core;
disposing at least the absorbent core between the top sheet and the back sheet to form a composite sheet, the composite sheet having a longitudinal dimension and a lateral dimension, whereby at least the top sheet, back sheet, and absorbent core are prepared and arranged such that the top sheet and the back sheet form a first waist region having a rear distal edge in the lateral direction, a second waist region having a front distal edge in the lateral direction longitudinally opposite the first waist region, and a crotch region between the first and second waist regions;
partially dissociating at least some of the composite sheet in the longitudinal dimension to form at least one lateral side portion, whereby the lateral side portion has an inner distal edge, an outer distal edge, a rear longitudinal edge, and a front longitudinal edge, the inner distal edge situated closer to the longitudinal centerline of the composite sheet than the outer distal edge and the rear longitudinal edge situated closer to the first waist region than the front longitudinal edge;
folding the at least one lateral side portion to form at least one side panel, whereby one of the outer distal edge or the inner distal edge of the at least one lateral side portion is substantially coterminous with the rear distal edge of the first waist region to form a rear waist edge;
disposing at least one fastening mechanism on the at least one lateral side panel; and
disposing one or more target devices in the second waist region,
whereby the rear waist edge while in a relaxed state is capable of having a greater lateral dimension than the lateral dimension of the composite sheet.

16. The method of claim 15, wherein the at least one lateral side panel comprises at least one of the top sheet and back sheet.

17. The method of claim 15, wherein the length of the rear longitudinal edge is substantially equal to or greater than the length of the front longitudinal edge.

18. The method of claim 15, wherein the at least one fastening element comprises a hook portion of a hook and loop fastener and the one or more target devices comprise the loop portion of a hook and loop fastener.

19. The method of claim 18, wherein at least one of the hook or loop portion are integrally formed from a layer of the composite sheet.

20. The method of claim 15, wherein the at least one fastening element comprises an adhesive tape and the one or more target devices comprise a tape receiving surface.

21. The method of claim 15, further comprising disposing elastic elements in either a longitudinal or a longitudinal and lateral direction between the top sheet and the back sheet such that the elastic elements are disposed between the inner and outer distal edges of the lateral side portion.

22. The method of claim 21, wherein the elastic elements comprise elastic webbing, netting, scrim elastic, or strands or bands of elastic materials or combinations thereof.

23. The method of claim 22, wherein the strands or bands of elastic materials comprise natural or synthetic rubber, urethane elastomers, spandex, LYCRA, elastic polymers, and mixtures thereof.

24. The method of claim 21, wherein the elastic elements comprise about 2 to about 20 strands or bands of elastic materials.

25. The method of claim 25, wherein the strands or bands of elastic materials are partially associated with the at least one side panel such that the elasticity of the at least one side panel is varied across its lateral dimension.

26. The method of claim 15, wherein the at least one lateral side panel comprises two separate panels.

27. The method of claim 26, wherein the two separate panels comprise a first material and a second material.

28. The method of claim 27, wherein the first material comprises the back sheet material, and the second material comprises the top sheet material.
